(19) United States
(12) Patent Application Publication
(10) Pub. No.: US 2024/0115646 A1
(43) Pub. Date: Apr. 11, 2024

(54) Nutritional Supplement

(71) Applicant: GlaxoSmithKline Consumer Healthcare Holdings (US) LLC, Wilmington, DE (US)

(72) Inventors: Nadine Aubourg, Richmond, VA (US); Kamala Arango, Richmond, VA (US)

(21) Appl. No.: 18/045,490

(22) Filed: Oct. 11, 2022

(51) Int. Cl. A61K 36/73; A61K 9/00; A61K 31/375; A61K 31/519; A61K 31/593; A61K 33/30; A61K 33/32; A61K 47/26; A61K 47/36

(52) U.S. Cl. CPC A61K 36/73; A61K 9/0056; A61K 31/375; A61K 31/519; A61K 31/593; A61K 33/30; A61K 33/32; A61K 47/26; A61K 47/36; A61K 2236/19

(57) ABSTRACT

A composition comprises about 1% to 5% by weight of apple cider vinegar; a gellant; about 1% to 10% by weight ascorbic acid; a sweetener; and water.

### TECHNICAL FIELD

The invention is generally related to nutritional supplement compositions, and, more specifically, to gummy nutritional supplement compositions.

### BACKGROUND

Apple cider vinegar (“ACV”) is generally produced from the juice of crushed apples by fermenting the juice with a yeast to convert the natural sugars into alcohol. A second fermentation is then performed using a bacteria that converts that alcohol into acetic acid, which gives ACV its distinctive sharp, sour taste. The health benefits of ACV have long been recognized in ancient medicine, with ACV being used to treat everything from digestion issues to coughs and colds. More recently, ACV has been rediscovered as health aid, with studies exploring the effectiveness in losing weight, increasing mineral absorption, and controlling diabetes, to name a few examples.

Despite the recognized health benefits of ACV, the sour taste, high acidity, and distinct smell of liquid ACV have been a limiting factor in widespread consumption of ACV. While various dosage forms have been explored to mask the undesirable taste, smell, and acidity of ACV, these efforts have only been marginally successful. Moreover, many of these dosage forms experience problematic physical properties. For instance, ACV-containing gummies often experience excessive “sweating”, stickiness, and other undesirable physical properties.

Accordingly, there is a need for improved dosage forms of ACV that alleviate or reduce the undesirable taste, smell, and/or acidity while maintaining the health benefits, and a need for improved physical properties of the dosage forms.

### SUMMARY

In one aspect, a gummy composition comprises about 1% to 5% by weight of apple cider vinegar; a gellant; 1% to 10% by weight ascorbic acid; a sweetener; and water. In some embodiments, the composition comprises about 1% to 3% by weight of apple cider vinegar. In some cases, the composition comprises about 2%, by weight of apple cider vinegar.

In some instances, a gellant comprises a pectin, a gelatin, a gum, a seaweed, a starch, a lecithin, or any combination thereof. The composition can comprise about 0.1% to 6% by weight of gellant. In some embodiments, the gellant comprises pectin. The pectin can be present in the composition in an amount of about 1% to 6% by weight.

In some instances, the composition comprises ascorbic acid (vitamin C). The ascorbic acid can be present in the composition in an amount of about 1% to 10% by weight, or in some cases, in an amount of about 2% to 5% by weight.

Compositions described herein can further comprise one or more vitamins, minerals, or any combination thereof in some embodiments. Exemplary vitamins and minerals can comprise a vitamin B, a zinc, a manganese, a vitamin D, or any combination thereof, and/or any pharmaceutically acceptable salt thereof.

In some embodiments, the composition can comprise about 50% to 85% by weight of sweetener. In some embodiments, the sweetener can comprise a monosaccharide, a disaccharide, a polysaccharide, or any combination thereof. Exemplary monosaccharides can comprise fructose, glucose, dextrose, or any combination thereof. In some instances, the monosaccharide comprises glucose. Exemplary disaccharides comprise sucrose, cane sugar, beet sugar, evaporated cane juice, cane crystals, or any combination thereof. In some cases, the disaccharide is cane sugar. The total sweetener present in the composition can be comprised of about 30%-60% by weight of cane sugar. Exemplary polysaccharides can comprise a glucose syrup, a corn syrup, an agave syrup, a high maltose syrup, a fruit concentrate, or any combination thereof. The total sweetener present in the composition can be comprised of about 10% to 90% by weight of the polysaccharide.

In some embodiments, the composition can further comprise a pH modifier, a flavorant, a diluent, a colorant, a preservative, or any combination thereof.

In some embodiments, a composition can comprise about 1% to 3% by weight of apple cider vinegar; pectin; 2% to 5% by weight ascorbic acid; one or more sweeteners; and water. The composition can further comprise a vitamin B, a zinc, a magnesium, D vitamin, or any combination thereof in some cases.

### BRIEF DESCRIPTION OF THE DRAWINGS

FIG. 1 is a flowchart showing a method of preparing ACV-containing gummies having compositions described herein.

FIG. 2 is a photograph of a commercially available gummy having approximately 10-12% by weight ACV.

FIG. 3 is a photograph of gummies according to compositions described herein having 2% by weight ACV.

### DETAILED DESCRIPTION

Embodiments described herein can be understood more readily by reference to the following detailed description, examples, and figures. Elements, apparatus, and methods described herein, however, are not limited to the specific embodiments presented in the detailed description, examples, and figures. It should be recognized that the exemplary embodiments herein are merely illustrative of the principles of the present invention. Numerous modifications and adaptations will be readily apparent to those of skill in the art without departing from the spirit and scope of the invention.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should generally be considered to include the end points 5 and 10.

The unit of “percentage by weight” is interchangeably represented as “% by weight” or “wt. %” unless expressly stated otherwise.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

A composition suitable as a nutritional supplement is described herein. The composition is an edible composition comprising apple cider vinegar (ACV) and ascorbic acid (Vitamin C), and can in some embodiments, achieve or alleviate one or more deficiencies found in other edible compositions having ACV. For example, it has been found that gummy compositions having a combination of ascorbic acid and an ACV content greater than 5% by weight exhibit undesirable physical and organoleptic properties. For example, in gummy compositions having 6% by weight or greater ACV can form gummies that “sweat”, a highly undesirable property for gummies because it causes short and long-term storage problems. Sweating gummies stick together in a container to form a congealing mass that has to be manually shaken or physically pulled apart to separate individual gummies. In some cases, the sweating gummies can permanently stick together and are unable to be separated back into the original individual gummies—ruining the product.

The higher amounts of ACV can also have undesirable organoleptic properties. For instance, gummies that have 6% by weight or higher ACV have a strong vinegar smell and taste that can be unpleasant to a user. Additionally, the higher amounts of ACV in combination with ascorbic acid make the gummies more acidic, which can affect the tooth enamel of a user.

As described in more detail herein, the following compositions comprise about 1% to 5% by weight of apple cider vinegar; a gellant; 2% to 5% by weight ascorbic acid; one or more sweeteners; and water. In some embodiments, the compositions comprise about 1% to 3% by weight of apple cider vinegar; a gellant; 2% to 5% by weight ascorbic acid; one or more sweeteners; and water. These compositions can further comprise a vitamin B, a zinc, a magnesium, D vitamin, or any combination of vitamins and/or minerals. Furthermore, these embodiments can further comprise additional excipients as previously described herein, such as a pH modifier, a flavorant, a diluent, a colorant, a preservative, or any combination thereof.

### I. Compositions

In an aspect, a composition comprises apple cider vinegar (“ACV”), a gellant, ascorbic acid, and water. In some embodiments, the composition is a chewable dosage form, such as a gummy, a bar, or a confection.

Compositions described herein comprise about 1% to 5% by weight of ACV. It has been found that compositions with amounts of ACV greater than about 5% by weight exhibit undesirable organoleptic properties, such as excessive sourness and strong ACV odors. Moreover, higher amounts of ACV correspond to higher acidity, which has a negative effect on the physical properties of the composition and on a user, such as a user's tooth enamel. In some embodiments, compositions described here comprise about 1%-3% by weight of ACV. In some cases, composition described here comprise about 2% by weight of ACV. The ACV used in the composition is generally the unfiltered mother liquid produced from the fermentation of apple cider, which can include the enzymes, proteins, bacteria, etc., used and created during the fermentation process.

Further exemplary amounts of ACV in the composition include about 1%-5%, 1%-4.8%, 1%-4.5%, 1%-4.3%, 1%-4%, 1%-3.8%, 1%-3.5%, 1%-3.3%, 1%-3%, 1%-2.8%, 1%-2.5%, 1%-2.3%, 1%-2%, 1%-1.8%, 1%-1.5%, 1%-1.3%, 1.3%-5%, 1.5%-5%, 1.8%-5%, 2%-5%, 2.3%-5%, 2.5%-5%, 1.8%-5%, 3%-5%, 3.3%-5%, 3.5%-5%, 3.8%-5%, 4%-5%, 4.3%-5%, or 4.5%-5%, 1%, 1.3%, 1.5%, 1.8%, 2%, 2.3%, 2.5%, 2.8%, 3%, 3.2%, 3.5%, 3.8% 4%, 4.3%, 4.5%, 4.8% or 5% by weight.

Compositions herein can comprise a gellant. Exemplary gellants include a pectin, a gelatin, a gum, a seaweed, a starch, a lecithin or any combination thereof. In some embodiments, the gellant is a pectin and/or a gelatin. In some instances, the gellant is pectin. The skilled artisan would recognize that the amount of gellant present in the composition will vary based on the physical characteristics of the particular gellant being used, the desired consistency, and the dosage form (such as a gummy). In some cases, the gellant can be present in any amount appropriate to make a gummy dose form. In some embodiments, the composition comprises about 0.1% to 6% by weight of gellant. In some cases, the composition comprises about 0.1% to 5.8%, 0.1% to 5.5%, 0.1% to 5.3%, 0.1% to 5%, 0.1 to 4.8%, 0.1% to 4.5%, 0.1% to 4.3%, 0.1% to 4%, 0.1% to 3.8%, 0.1% to 3.5%, 0.1% to 3.3%, 0.1% to 3%, 0.1% to 2.8%, 0.1% to 2.5%, 0.1% to 2.3%, 0.1% to 2%, 0.1% to 1.8%, 0.1% to 1.5%, 0.1% to 1.3%, 0.1% to 1%, 0.1% to 0.8%, 0.1% to 0.5%, 0.1% to 0.3%, 0.3% to 6%, 0.5% to 6%, 0.8% to 6%, 1% to 6%, 1.3% to 6%, 1.5% to 6%, 1.8% to 6%, 2% to 6%, 2.3% to 6%, 2.5% to 6%, 2.8% to 6%, 3% to 6%, 3.3% to 6%, 3.5% to 6%, 3.8% to 6%, 4% to 6%, 4.3% to 6%, 4.5% to 6%, 4.8% to 6%, 5% to 6%, 5.3% to 6%, 5.5% to 6%, 0.3% to 3%, 0.5% to 2.8%, 0.8% to 2.5%, 1% to 2.3%, 1.3% to 2%, 0.3% to 2%, 0.5% to 1.8%, 6%, 5.8%, 5.5%, 5.3%, 5%, 4.8%, 4.5%, 4.3%, 4%, 3.8%, 3.5%, 3.3%, 3.0%, 2.8%, 2.5%, 2.3%, 2%, 1.8%, 1.5%, 1.3%, 1%, 0.8%, 0.5%, 0.3%, or 0.1% by weight of gellant.

A pectin can be any pectin not inconsistent with the objectives of this disclosure. Exemplary pectin sources can include apples (such as apple pomace), pears, berries, citrus fruits (such as from citrus peels), or any combination thereof.

In some embodiments, the composition comprises about 1% to 6% by weight pectin. In some instances, the composition comprises about 1% to 5.8%, 1% to 5.5%, 1% to 5.3%, 1% to 5%, 1% to 4.8%, 1% to 4.5%, 1% to 4.3%, 1% to 4%, 1% to 3.8%, 1% to 3.5%, 1% to 3.3%, 1% to 3%, 1% to 2.8%, 1% to 2.5%, 1% to 2.3%, 1% to 2%, 1% to 1.8%, 1% to 1.5%, 1.3% to 6%, 1.5% to 6%, 1.8% to 6%, 2% to 6%, 2.3% to 6%, 2.5% to 6%, 2.8% to 6%, 3% to 6%, 3.3% to 6%, 3.5% to 6%, 3.8% to 6%, 4% to 6%, 4.3% to 6%, 4.5% to 6%, 4.8% to 6%, 5% to 6%, 5.3% to 6%, 1.3% to 5.8%, 1.5% to 5.5%, 1.8% to 5.3%, 2 to 5%, 2.3% to 4.8%, 2.5% to 4.5%, 6%, 5.8%, 5.5%, 5.3%, 5%, 4.8%, 4.5%, 4.3%, 4%, 3.8%, 3.5%, 3.3%, 3.0%, 2.8%, 2.5%, 2.3%, 2%, 1.8%, 1.5%, 1.3%, or 1% by weight of pectin.

The gelatin can be any gelatin not inconsistent with the objectives of this disclosure. Exemplary gelatin sources can include bovine, porcine, or fish.

The gum can be any gum not inconsistent with the objectives of this disclosure. For example, the gum can include acacia, gellan, guar, locust bean, tara, xanthan, gum Arabic, or any combination thereof.

The seaweed can be any seaweed or seaweed extract not inconsistent with the objectives of this disclosure. For instance, examples of seaweed include an agar and/or a carrageenan. In some embodiments, the seaweed can be present in any amount appropriate to make a gummy dose form.

The starch can be any starch not inconsistent with the objectives of this disclosure. Examples of starch sources include corn, tapioca, potato, rice or any combination thereof.

Compositions described herein comprise ascorbic acid in some instances. In some embodiments, the composition comprises about 1% to 10% by weight of ascorbic acid. In other instances, the composition comprises about 1 to 9.8%, 1 to 9.6%, 1 to 9.4%, 1 to 9.2%, 1 to 9%, 1 to 8.8%, 1 to 8.6%, 1 to 8.4%, 1 to 8.2%, 1 to 8%, 1 to 7.8%, 1 to 7.6%, 1 to 7.4%, 1 to 7.2%, 1 to 7%, 1 to 6.8%, 1 to 6.6%, 1 to 6.4%, 1 to 6.2%, 1 to 6%, 1 to 5.8%, 1 to 5.6%, 1 to 5.4%, 1 to 5.2%, 1 to 5%, 1 to 4.8%, 1 to 4.6%, 1 to 4.4%, 1 to 4.2%, 1 to 4%, 1 to 3.8%, 1 to 3.6%, 1 to 3.4%, 1 to 3.2%, 1 to 3%, 1 to 2.8%, 1 to 2.6%, 1 to 2.4%, 1 to 2.2%, 1 to 2%, 1 to 1.8%, 1 to 1.6%, 1 to 1 to 1.4%, 1.2 to 10%, 1.4 to 10%, 1.6 to 10%, 1.8 to 10%, 2 to 10%, 2.2 to 10%, 2.4 to 10%, 2.6 to 10%, 2.8 to 10%, 3 to 10%, 3.2% to 10%, 3.4 to 10%, 3.6 to 10%, 3.8 to 10%, 4 to 10%, 4.2 to 10%, 4.4 to 10%, 4.6 to 10%, 4.8 to 10%, 5 to 10%, 5.2 to 105, 5.4 to 10%, 5.6 to 10%, 5.8 to 10%, 6 to 10%, 6.2 to 10%, 6.4 to 10%, 6.6 to 10%, 6.8 to 10%, 7 to 10%, 7.2 to 10%, 7.4 to 10%, 7.6 to 10%, 7.8 to 10%, 8 to 10%, 8.2 to 10%, 8.4 to 10%, 8.6 to 10%, 8.8 to 10%, 9 to 10%, 9.2 to 10%, 9.4 to 10%, 9.6 to 10%, 1.5 to 9.5%, 2 to 9%, 2.5 to 8.5%, 3 to 8%, 3.5 to 7.5%, 4 to 7%, 4.5 to 6.5%, 2% to 8%, 2% to 7%, 2% to 5%, 2% to 4%, 2% to 3%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of ascorbic acid.

In some embodiments, compositions described here comprise a sweetener. The sweetener can comprise a monosaccharide, a disaccharide, a polysaccharide, or any combination thereof. The amount of sweetener present in the composition can be any amount not inconsistent with the objectives of this disclosure. For example, in some cases the composition can comprise 50% to 85% by weight of sweetener. In some instances, the composition comprises 55% to 85%, 60% to 85%, 65% to 85%, 70% to 85%, 75% to 85%, 80% to 85%, 50% to 80%, 50% to 75%, 50% to 70%, 50% to 65%, 50% to 60%, 50% to 55%, 55% to 75%, 60% to 70, 50%, 55%, 60%, 65%, 70%, 75%, 80%, or 85% by weight of sweetener.

A monosaccharide described herein can comprise fructose, glucose, dextrose, or any combination thereof. The amount of monosaccharide present in the composition can be any amount not inconsistent with the objectives of this disclosure. When present in the composition, the total sweetener in the composition can comprise about 1% to 90% by weight of monosaccharide. In some cases, the total sweetener in the composition comprises about 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 90%, 10% to 90%, 15% to 90%, 20% to 90%, 25% to 90%, 30% to 90%, 35% to 90%, 40% to 90%, 45% to 90%, 50% to 90%, 55% to 90%, 60% to 90%, 65% to 90%, 70% to 90%, 75% to 90%, 80% to 90%, 5% to 85%, 10% to 80%, 15% to 75%, 20% to 70%, 25% to 65%, 30% to 60%, 35% to 55%, 40% to 50%, 1%, 5%, 10%, 15%, 20%, 25%, 30%, 35%, 40%, 45%, 50%, 55%, 60%, 65%, 70%, 75%, 80%, 85%, or 90% by weight of monosaccharide.

A disaccharide described herein can comprise sucrose, including cane sugar, beet sugar, evaporated cane juice, cane crystals, or any combination thereof. In some embodiments, the disaccharide is cane sugar. The amount of disaccharide present in the composition can be any amount not inconsistent with the objectives of this disclosure. In instances where the disaccharide is cane sugar, the total sweetener in the composition can comprise about 30%-60% by weight of cane sugar. In some cases, the total sweetener in the composition comprises about 30% to 58%, 30% to 55%, 30% to 53%, 30% to 50%, 30% to 48%, 30% to 45%, 30% to 43%, 30% to 40%, 30% to 38%, 30% to 35%, 33% to 60%, 35% to 60%, 38% to 60%, 40% to 60%, 43% to 60%, 45% to 60%, 48% to 60%, 50% to 60%, 53% to 60%, 55% to 60%, 30%, 33%, 35%, 38%, 40%, 43%, 45%, 48%, 50%, 53%, 55%, 58%, or 60% by weight of disaccharide.

A polysaccharide described herein can comprise a glucose syrup, a corn syrup (such as a high fructose corn syrup and/or a corn syrup), an agave syrup, a high maltose syrup, a fruit concentrate, or any combination thereof. The amount of polysaccharide present in the composition can be any amount not inconsistent with the objectives of this disclosure. For example, in some embodiments, the total sweetener in the composition comprises about 10% to 80% by weight of the polysaccharide. In some cases, the total sweetener in the composition comprises about 10% to 75%, 10% to 70%, 10% to 65%, 10% to 60%, 10% to 55%, 10% to 50%, 10% to 45%, 10% to 40%, 10% to 45%, 10% to 40%, 10% to 35%, 10% to 30%, 10% to 25%, 10% to 20%, 10% to 15%, 15% to 80%, 20% to 80%, 25% to 80%, 30% to 80%, 35% to 80%, 40% to 80%, 45% to 80%, 50% to 80%, 55% to 80%, 60% to 80%, 65% to 80%, 70% to 80%, 15% to 60%, 20% to 55%, 25% to 50%, 30% to 45%, 10%, 15%, 20%, 25%, 30%, 35%, 40%, 45%, 50%, 55%, 60%, 65%, 70%, 75%, or 80% by weight of polysaccharide.

It is also contemplated that artificial sweeteners can be used in some embodiments, either alone or in combination with the natural sweeteners. Any artificial sweetener(s) known to the skilled artisan can be used. The skilled artisan would recognize that the amount of artificial sweetener present in the composition will vary depending on the physical characteristics of the artificial sweetener being used, and the desired sweetness and physical properties of the composition.

In some embodiments, compositions described herein can further comprise additional vitamins, minerals, or any combination thereof. Exemplary vitamins can comprise a vitamin B (B3, B6, B9, B12, and biotin), vitamin E, vitamin A, vitamin D, or any combination thereof. Exemplary minerals can comprise a zinc, a manganese, a magnesium, or any combination thereof.

Compositions described herein can further comprise additional excipients, such as a pH modifier, a flavorant, a diluent, a colorant, a preservative, or any combination thereof.

In some embodiments, a pH modifier (buffering agent) is present in the composition. Exemplary pH modifiers include a sodium citrate, citric acid, acetic acid, maleic acid, tartaric acid, sodium acetate, sodium hydroxide, or any combination thereof, although any pH modifier not inconsistent with the objectives of this disclosure can be used. The quantity of pH modifiers present in the composition is dependent on the physical properties of the pH modifier, such as the pKa, the desired final pH of the composition, and the like.

Optionally, a flavorant can be used in the composition. When present, the flavorant can be any flavorant known in the art that is not inconsistent with the objectives of this disclosure. As known by the skilled artisan, the amount of flavorant present will depend on the desired flavor, the specific flavorant being used, and the dosage form of the composition.

In some embodiments, a diluent can be used in the composition. The diluent can be any diluent known in the art that is not inconsistent with the objectives of this disclosure. As known by the skilled artisan, the amount of diluent present will depend on the desired physical properties of the composition, the specific diluent being used, and the dosage form of the composition. An example of a diluent includes water.

Optionally, a colorant can be used in the composition. When present, the colorant can be any colorant known in the art that is not inconsistent with the objectives of this disclosure. As known by the skilled artisan, the amount of colorant present will depend on the desired color, the specific colorant being used, and the dosage form of the composition.

In some instances, a preservative is present in the composition. The preservative can be any preservative not inconsistent with the objectives of this disclosure. Exemplary preservatives include sodium benzoate, benzyl alcohol, methyl paraben, propyl paraben, or any combination thereof. The preservative can be present in the composition in amounts up to 2 wt. %, up to 1.8 wt. %, up to 1.6 wt. %, up to 1.4 wt. %, up to 1.2 wt. %, up to 1 wt. %, up to 0.8 wt. %, up to 0.6 wt. %, up to 0.4 wt. %, up to 0.2 wt. %, or up to 0.1 wt. %.

Finally, compositions described herein can comprise water. Typically, in most cases, the balance of the composition is comprised of water. For example, water can be present as the balance remaining in the % by weight of the composition.

### II. Methods

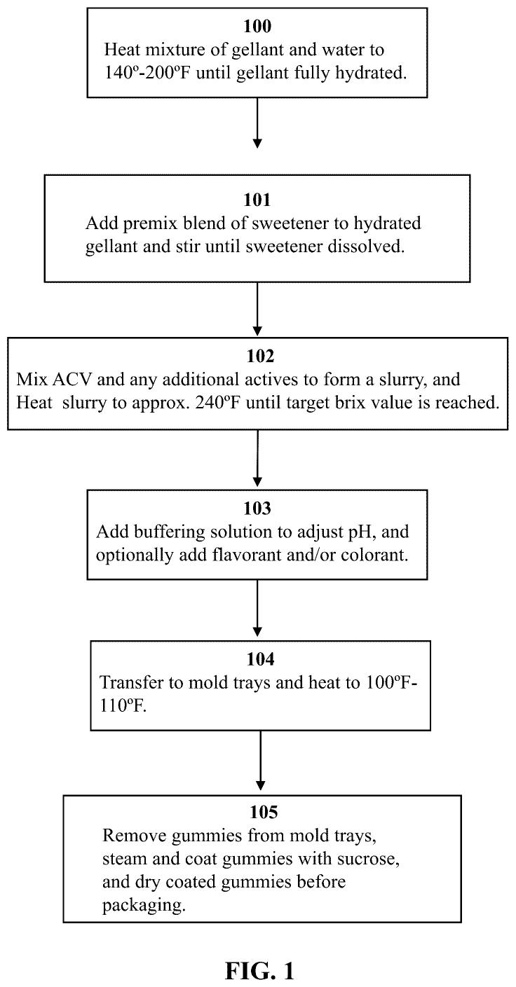

In another aspect, methods of preparing compositions disclosed in Section I herein are described. As illustrated in FIG. 1, a composition of Section I can be prepared by mixing a gellant in water at a temperature of approximately 100°-200° F. until the pectin becomes fully hydrated and translucent, forming an aqueous gellant mixture at step 100. A premix blend of sweetener is added to the heated aqueous gellant mixture and stirred until the sweeteners have dissolved at step 101. ACV is then added to the aqueous gellant/sweetener mixture to form a slurry. Optionally, additional actives, such as vitamins and/or minerals can be added at the same time or after ACV is added to form the slurry. The resulting slurry is heated to approximately 240° F. until a target brix is reached at step 102. When the slurry reaches the target brix, a buffering solution of aqueous citric acid is added to adjust the pH to a desired range at step 103. Optionally, flavoring and/or colorants can be added at step 103 as well. Once the desired pH is reached, the composition is transferred to starch mold trays. The trays are then heated between approximately 100° F.-110° F. for at least 12 hours before being removed at step 104. The resulting gummies are steamed and coated in sucrose before being dried for approximately 24 hours at step 105. The dried, coated gummies are then transferred into bottles or other packaging. It is noted that the temperatures described in this method are approximate, and that the skilled artisan would recognize that the exact temperature can deviate from the listed temperatures in some cases.

In an embodiment where a composition described in Section I comprises ACV, pectin as a gellant, ascorbic acid, one or more sweeteners, and water, the composition can be prepared by mixing pectin with hot water at approximately 140° F.-194° F. until fully hydrated and translucent to form an aqueous pectin solution. A sweetener premix is prepared by heating a syrup (such as corn syrup) with additional monosaccharide, disaccharide, and/or oligosaccharide sweeteners. The aqueous pectin solution is then added to the sweetener premix and mixed until well blended. ACV, and optionally any other desired actives (such as vitamins, minerals, and/or any other nutritional or active ingredient premix), is then added to the aqueous gellant/sweetener mixture. The temperature is then raised to approximately 240° F. and cooked until the resulting slurry reaches a target brix. A premix buffering solution of 50% of citric acid and water is prepared, and when the slurry reaches the brix, the premix citric acid solution is added, along with any desired flavors and colors until a desired pH is reached. The composition is then transferred into starch mold trays and warmed at approximately 104° F. for at least 12 hours. The gummies are then removed from starch trays. The gummies are then lightly steamed before being coated in sucrose. Excess sucrose is removed and then the gummies are dried for about 24 hours before packaging.

### Example 1

### Commercial Gummy Composition with 10-12% by Weight ACV

A commercially available pectin-based gummy having approximately 10-12% by weight ACV content was purchased from a local pharmacy, and the physical characteristics of the gummy were examined. The commercial gummy is believed to be covered by U.S. Pat. No. 10,980,266, and according to the product label, the commercial gummies were 6 months old. Table 1 shows the ingredient contents listed on the label, where a serving size of 2 commercial gummies weighs approximately 8-10 g.

TABLE 1
Commercial Gummy Composition

| INGREDIENT | QUANTITY |
|---|---|
| Total Carbohydrate | 8 g |
| Folate (Folic Acid | 400 mcg DFE |
| | (240 mcg folic acid) |
| Vitamin B12 (Cyanocobalamin) | 2.4 mcg |
| Apple Cider Vinegar (5% Acetic Acid) | 1000 mg |

Additional ingredients listed on the commercial label are organic cane sugar, organic tapioca syrup, pectin, water, citric acid, malic acid, natural apple flavor, organic fruit and vegetable juice (for color), sodium citrate, organic pomegranate powder, and organic beet root powder.

As previously discussed herein, gummy compositions having ACV levels of ≥6% by weight have been observed to have undesirable organoleptic and physical properties, such as a strong vinegar smell and taste, excessive “sweating,” and stickiness that results in gummies clumping together into masses that are difficult to separate. Consistent with these observations, the commercial gummies exhibited each of these undesirable properties.

Firstly, the commercial gummies had a strong, unpleasant vinegar taste that was unable to be masked by the flavorants used in the composition. This unpleasant taste was compounded by a strong vinegar smell.

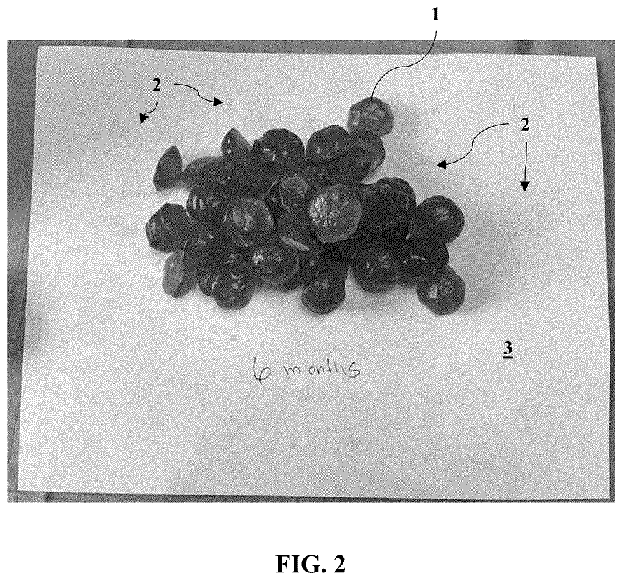

In addition to the undesirable organoleptic properties, the commercial gummies exhibited a high amount of “sweating” and were very sticky when touched. FIG. 2 shows the commercial gummies 1 after being poured out onto a sheet of paper 3. Smear marks 2 where the liquid/syrupy “sweat” can be readily observed on the sheet of paper 3 anywhere a commercial gummy 1 made contact. Further, when touched, each commercial gummy had a slimy, sticky surface. Moreover, as shown in the FIG. 2, the commercial gummies 1 were difficult to remove from their container onto the sheet of paper 3 because their sticky surface caused the gummies to stick together into a big clump or mass.

In summary, commercially available gummy formulations having 10-12% by weight ACV display unpleasant organoleptic properties, exhibit excessive and undesirable “sweating”, and stick together to form clumps/masses that are not easily separable, as seen in FIG. 2.

### Example 2

### 2% to 5% by Weight ACV-Containing Compositions

A series of gummy compositions were prepared having about 1% to 5% by weight of apple cider vinegar; pectin; 2% to 5% by weight ascorbic acid; one or more sweeteners; and water. Specifically, Table 2 lists the ingredients prepared for gummy compositions 1-3.

TABLE 2
Gummy Compositions with 1%-5% by weight ACV

| | TOTAL | ASCORBIC | |
|---|---|---|---|
| SAMPLE | CARBOHYDRATE | ACID | ACV |
| Composition 1 | 14 g | 250 mg | 2 wt. % |
| Composition 2 | 14 g | 250 mg | 4 wt. % |
| Composition 3 | 14 g | 250 mg | 5 wt. % |

Additional ingredients used in the gummy composition include sugar, glucose syrup, water, citric acid, natural flavor, a vitamin B mix, organic purple carrot juice (color), pectin, and sodium citrate.

Compositions 1-3 were prepared according to the method described in Section II herein. Specifically, compositions 1-3 were prepared by mixing approximately 393 g of pectin with hot water at approximately 140° F.-194° F. until fully hydrated and translucent to form an aqueous pectin slurry. A sweetener premix was prepared by combining 6289 g of corn syrup and 6417 g of sucrose and mixing at 104° F. The pectin slurry was then added to the sweetener premix and mixed until well blended. Approximately 78 g of sodium citrate, ACV mother liquid, 425 g of ascorbic acid, and 34 g of a Vitamin B premix were added to the pectin slurry/sweetener mixture and cooked at 240° F. until the desired brix was reached. For 2% by weight ACV Composition 1, approximately 300 g of ACV mother liquid was added. For 4% by weight ACV Composition 2, approximately 600 g of ACV mother liquid was added. For 5% by weight ACV Composition 3, approximately 750 g of ACV mother liquid was added. Approximately 166 g of citric acid solution (50/50) was then added to bring the pH of the mixture to a pH of approximately 3-4. Colorants and Flavorants were then added, and the resulting mixture heated to 160-190° F. The temperature is then raised to approximately 240° F. and cooked until the resulting slurry reaches a target brix. The composition is then transferred into starch mold trays and warmed at approximately 104° F. for at least 12 hours. The gummies are then removed from starch trays. The gummies are then lightly steamed before being coated in sucrose. Excess sucrose is removed and then the gummies are dried for about 24 hours before packaging.

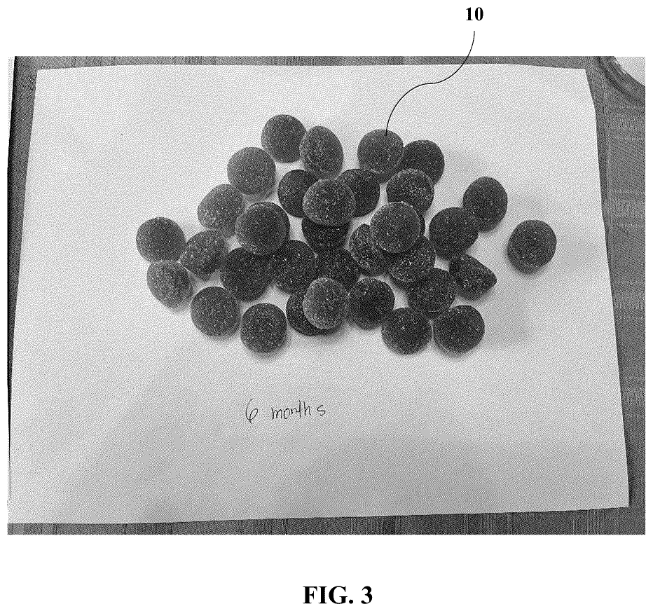

FIG. 3 shows pectin-based gummies having Composition 1 with 2% by weight (wt. %) ACV after 6 months at room temperature. In contrast to the 6 month old commercial gummies discussed in Example 1 and shown in FIG. 2, Composition 1 gummies did not have a strong vinegar odor and taste. Additionally, no “sweating” was observed, which meant that the gummies were easily poured out onto the paper 2 as individual pieces, did not have a “slimy” surface, and did not clump together to form a mass (as seen in FIG. 2).

Gummies made by Compositions 2 and 3 having 4% by weight and 5% by weight of ACV, respectively, showed the same or similar organoleptic and physical properties as the gummies having Composition 1.

Consequently, it has been discovered that high levels of ACV (>5% by weight) form gummies with undesirable organoleptic and physical properties and poor storage properties, whereas gummies with 1%-5% by weight ACV form gummies that do not suffer from those undesirable traits and properties.

1. A gummy composition comprising:
about 1.0% to 5.0% by weight of apple cider vinegar;
a gellant;
about 1% to 10% by weight ascorbic acid;
a sweetener; and
water.

2. A The gummy composition of claim 1, comprising 1% to 3% by weight of apple cider vinegar.

3. A The gummy composition of claim 1, comprising 2%, by weight of apple cider vinegar.

4. A The gummy composition of claim 1, wherein the gellant comprises a pectin, a gelatin, a gum, a seaweed, a starch, a lecithin, or any combination thereof.

5. A The gummy composition of claim 4, comprising about 0.1% to 6% by weight of gellant.

6. A The gummy composition of claim 1, where the gellant comprises pectin.

7. A The gummy composition of claim 6, comprising about 1% to 6% by weight pectin.

8. A The gummy composition of claim 1 comprising 2% to 5% by weight ascorbic acid.

9. A The gummy composition of claim 1 further comprising one or more vitamins, minerals, or any combination thereof.

10. A The gummy composition of claim 9, wherein the vitamins and minerals comprise a vitamin B, a zinc, a manganese, a vitamin D, or any combination thereof.

11. A The gummy composition of claim 1, wherein the sweetener can comprise a monosaccharide, a disaccharide, a polysaccharide, or any combination thereof, the total sweetener being present in the composition in an amount of about 50% to 85% by weight.

12. A The gummy composition of claim 11, wherein the monosaccharide comprises fructose, glucose, dextrose, or any combination thereof.

13. A The gummy composition of claim 11, wherein the disaccharide comprises sucrose, cane sugar, beet sugar, evaporated cane juice, cane crystals, or any combination thereof.

14. A The gummy composition of claim 13, wherein the disaccharide is cane sugar.

15. A The gummy composition of claim 14 wherein the total sweetener present in the composition comprises about 30%-60% by weight of cane sugar.

16. A The gummy composition of claim 11, wherein the polysaccharide comprises glucose syrup, a corn syrup, agave syrup, high maltose syrup, a fruit concentrate, or any combination thereof.

17. A The gummy composition of claim 16, wherein the total sweetener present in the composition comprises about 10% to 90% by weight of the polysaccharide.

18. A The gummy composition of claim 1, further comprising a pH modifier, a flavorant, a diluent, a colorant, a preservative, or any combination thereof.

19. A composition comprising:
about 1% to 3% by weight of apple cider vinegar;
pectin;
about 2% to 5% by weight ascorbic acid;
one or more sweeteners; and
water.

20. A The composition of claim 19 further comprising a vitamin B, a zinc, a magnesium, D vitamin, or any combination thereof.
